(19) United States
(12) Patent Application Publication
(10) Pub. No.: US 2011/0212120 A1
(43) Pub. Date: Sep. 1, 2011

(54) Ykur polynucleotides and polypeptides

(75) Inventors: Lloyd George Czaplewski, Oxfordshire (GB); Andrew Stephen Waller, Oxford (GB)

(73) Assignee: VERNALIS (OXFORD) LIMITED, OXFORD (GB)

(21) Appl. No.: 10/503,136

(22) PCT Filed: Feb. 11, 2003
(86) PCT No.: PCT/GB2003/000595

§ 371 Date: Mar. 31, 2005

(30) Foreign Application Priority Data
Feb. 13, 2002 (GB) 0203358.7

(51) Int. Cl. A61K 39/02; C07H 21/00; C12N 15/63; C12N 5/10; C12N 1/00; C12N 9/80; C12Q 1/68; C12Q 1/34; A61P 37/04; A61P 31/04

(52) U.S. Cl. 424/190.1; 536/23.2; 435/320.1; 435/325; 435/243; 435/419; 435/228; 435/6.18; 435/18; 424/184.1

(57) ABSTRACT

Nucleic acid encoding a novel bacterial polypeptide ykuR, the isolated ykuR polypeptide, its expression from host cells, and its use in screening for potential antibacterial agents. Inhibition of ykuR activity results in inhibition of bacterial growth.

This invention relates to isolated bacterial polynucleotides and polypeptides, and their production and uses, as well as their use as tools for the identification of antibacterial agents. In particular, the invention relates to bacterial ykuR polynucleotides and polypeptides.

### BACKGROUND TO THE INVENTION

The search for antibacterially active drugs has benefited from investigation of bacterial genomes to locate genes and gene products which are essential for normal growth or replication of the relevant bacterium. Since such genes and gene products are often shared by several different species of bacteria, compounds which disrupt the normal functions of those genes and gene products are often antibacterially active against a range of bacterial species, not only the species in which they were originally identified. Location of new antibacterial drug targets in this way is therefore a desirable objective, since it enables the discovery of new antibacterial agents, vaccines, and diagnostic tests for the presence of the relevant organism.

### BRIEF DESCRIPTION OF THE INVENTION

This invention is based on the identification of an open reading frame of the B. subtilis bacterial genome which defines a polynucleotide sequence designated ykuR encoding a putative metalloenzyme polypeptide sequence. Variants of the ykuR sequences have been found in other bacterial species. These sequences have, been found to be essential for normal growth of the organism, and thus represent excellent targets for antibacterial drug discovery. Such drugs would interfere with the normal expression of, or enzymic activity of, the polypeptide. Furthermore, the polypeptide, or immunogenic fragments thereof, could also prove useful as useful as antibacterial vaccines. In addition, the ykuR polynucleotide and polypeptide sequences have utility as diagnostic tools for bacterial infection and as research tools for identification of compounds which interfere with their normal activity.

It is therefore an object of the invention to provide ykuR polypeptides and polynucleotides, including mRNAs, cDNAs and genomic DNAs, which encode ykuR polypeptides.

It is also an object of the invention to make available isolated ykuR polynucleotides and polypeptides, for therapeutic, diagnostic and assay purposes, and for the purpose of screening libraries of chemical compounds for anti-ykuR polynucleotide and/or polypeptide activity.

### DETAILED DESCRIPTION OF THE INVENTION

The invention relates to isolated ykuR polynucleotides and polypeptides having the nucleotide and amino acid sequences set out in Table 1 as SEQ ID NO: 1 and SEQ ID NO: 2 respectively, and to subsequences and variants thereof.

TABLE 1

ykuR Polynucleotide Sequence [SEQ ID NO: 1]
5′-
ATGAAGATAGAGGAGCTCATCGCAATTCGCAGAGATCTGCATCGTATACCGGAGCT
TGGATTTCAGGAGTTCAAAACCCAGCAGTATTTATTAAATGTCTTGGAACAATATCC
GCAAGACAGAATTGAAATTGAGAAATGGCGAACAGGGCTTTTTGTAAAAGTGAACG
GGACGGCGCCGGAAAAAATGCTGGCATACAGAGCGGATATCGATGCGCTTTCTATA
GAAGAGCAAACTGGTCTTCCATTCGCATCAGAGCATCACGGCAACATGCACGCCTG
CGGTCACGATTTGCATATGACAATTGCACTCGGCATTATTGATCATTTTGTTCACCAC
CCAGTCAAACATGATTTGCTTTTTCTGTTTCAGCCGGCAGAGGAAGGGCCTGGCGGT
GCGGAACCAATGCTTGAGAGCGATGTATTAAAAAAATGGCAGCCTGATTTCATCAC
TGCCCTTCATATTGCTCCAGAGCTTCCGGTAGGCACCATTGCGACAAAAAGCGGCCT
TCTATTTGCGAATACATCAGAGCTAGTCATCGATCTGGAAGGCAAAGGGGGACATG
CGGCATATCCGCATTTGGCTGAGGATATGGTTGTAGCAGCAAGTACACTTGTCACCC
AGCTGCAAACGATTATCTCTAGAAACACAGATCCGCTAGACAGTGCTGTTATTACAG
TTGGTACCATTACCGGAGGCTCGGCACAAAATATCATTGCAGAAACGGCCCACCTG
GAAGGCACGATCCGCACGCTTTCTGAAGAATCGATGAAACAAGTAAAGGAACGGAT
TGAAGATGTAGTGAAAGGAATCGAAATCGGATTCCGCTGCAAAGGAAAAGTGACAT
ATCCGTCTGTATATCACCAAGTTTACAATACGAGCGGATTAACAGAAGAATTTATGT
CTTTTGTTGCTGAACATCAACTGGCGACAGTAATTGAAGCAAAAGAAGCAATGACT
GGAGAGGATTTTGGCTATATGCTGAAAAAATATCCCGGATTCATGTTCTGGCTCGGC
GCTGATTCTGAACATGGGCTTCATCATGCTAAGCTGAATCCCGATGAAAATGCGATA
GAAACAGCGGTTCATGTCATGACAGGTTATTTTTCTGTTTATGCCAAT
Polypeptide sequence [SEQ ID NO: 2].
NH2-
MKIEELIAIRRDLHRIPELGFQEFKTQQYLLNVLEQYPQDRIEIEKWRTGLFVKVN
GTAPEKMLAYRADIDALSIEEQTGLPFASEHHGNMHACGHDLHMTIALGIIDHFV
HHPVKHDLLFLFQPAEEGPGGAEPMLESDVLKKWQPDFITALHIAPELPVGTIAT
KSGLLFANTSELVIDLEGKGGHAAYPHLAEDMVVAASTLVTQLQTIISRNTDPLD
SAVITVGTITGGSAQNIIAETAHLEGTIRTLSEESMKQVKERIEDVVKGIEIGFRCK
GKVTYPSVYHQVYNTSGLTEEFMSFVAEHQLATVIEAKEAMTGEDFGYMLKKY
PGFMFWLGADSEHGLHHAKLNPDENAIETAVHVMTGYFSVYAN

In SEQ ID: 2, the metal binding motif, ie an enzymically active site, is underlined.

As used herein the term “subsequence” refers to a continuous sequence of at least 30 nucleic acids or at least 10 amino acids within a larger ykuR sequence, and which retains a biological activity of the larger sequence or retains an enzymically active site of the polypeptide. The polypeptides of the invention include a polypeptide of Table 1 [SEQ ID NO: 2] (in particular the mature polypeptide) as well as polypeptides and fragments, particularly those which have the biological activity of ykuR, and also those which have at least 70%, 80%, 85%, 90% or 95% identity to a polypeptide of Table 1 [SEQ ID NO: 1] or the relevant portion thereof.

As used herein the term “variant” refers to a sequence which preserves 50% or more, preferably 55% or more, more preferably 60% or more sequence homology (similarity) to Seq ID:1 or SEQ ID: 2. Variants include also those which have the amino acid sequence of ykuR polypeptide SEQ ID: 2, in which several, a few, 5 to 10, 1 to 5, 1 to 3, 2, or 1 amino acid residues are substituted, deleted or added, in any combination. Especially preferred among these are silent substitutions, additions and deletions, which have the biological activity of ykuR.

Using the polynucleotide sequence set out in Table 1 [SEQ ID NO: 1], an isolated polynucleotide of the invention encoding ykuR polypeptide may be obtained using standard cloning and screening methods, such as those for cloning and sequencing chromosomal DNA fragments from B. subtilis cells as starting material, followed by obtaining a full length clone. Suitable techniques are described by Maniatis, T., Fritsch, E. F. and Sambrook et al., MOLECULAR CLONING, A LABORATORY MANUAL, 2nd Ed.; Cold Spring Harbor Laboratory Press, Cold Spring Harbor, N.Y. (1989).

Polynucleotide and polypeptide sequences of the invention may be spliced to sequences performing functions other than ykuR activity. For example, polynucleotide sequences may be in reading frame with other coding sequence, such as those encoding a leader or secretory sequence, a pre-, or pro- or prepro-protein sequence. The polynucleotide may also contain non-coding sequences, including for example non-coding 5′ and 3′ sequences, such as the transcribed, non-translated sequences, termination signals, ribosome binding sites, sequences that stabilize mRNA, introns, polyadenylation signals, and additional coding sequence which encode additional amino acids. For example, a marker sequence that facilitates purification of the fused polypeptide can be encoded. In certain embodiments of the invention, the marker sequence is a hexa-histidine peptide, as provided in the pQE vector (Qiagen, Inc.) and described in Gentz et al., Proc. Natl. Acad. Sci., USA 86: 821-824 (1989), or an HA tag (Wilson et al., Cell 37: 767 (1984). Polynucleotides of the invention also include, but are not limited to, polynucleotides comprising a structural gene and its naturally associated sequences that control gene expression.

Thus, a polynucleotide of the invention may encode a mature protein, a mature protein plus a leader sequence (which may be referred to as a preprotein), a precursor of a mature protein having one or more prosequences that are not the leader sequences of a preprotein, or a preproprotein, which is a precursor to a proprotein, having a leader sequence and one or more prosequences, which generally are removed during processing steps that produce active and mature forms of the polypeptide.

Polynucleotide ykuR sequences, subsequences and variants of the invention include those which hybridize to SEQ ID:1 under stringent conditions. As herein used, the terms “stringent conditions” and “stringent hybridization conditions” mean hybridization will occur only if there is at least 95% and preferably at least 97% identity between the sequences. An example of stringent hybridization conditions is overnight incubation at 42° C. in a solution comprising: 50% formamide, 5×SSC (150 mM NaCl, 15 mM trisodium citrate), 50 mM sodium phosphate (pH7.6), 5×Denhardt's solution, 10% dextran sulfate, and 20 micrograms/ml denatured, sheared salmon sperm DNA, followed by washing the hybridization support in 0.1×SSC at about 65° C. Hybridization and wash conditions are well known and exemplified in Sambrook, et al., Molecular Cloning: A Laboratory Manual, Second Edition, Cold Spring Harbor, N.Y., (1989), particularly Chapter 11 therein.

Polynucleotides of the invention that are oligonucleotides derived from the SEQ ID NOS: 1 may be used in the processes herein as described, but preferably for PCR, to determine whether or not the polynucleotides identified herein in whole or in part are transcribed in bacteria in infected tissue. Such sequences will also have utility in diagnosis of the stage of infection and type of infection the pathogen has attained.

Polypeptides encoded by polynucleotide sequences of the invention may be expressed in host cells harbouring expression vectors comprising such sequences. Cell-free translation systems can also be employed to produce such proteins using RNAs derived from the DNA constructs of the invention.

For recombinant production, host cells can be genetically engineered to incorporate expression systems or portions thereof or polynucleotides of the invention. Introduction of a polynucleotide into the host cell can be effected by methods described in many standard laboratory manuals, such as Davis et al., Basic Methods in Molecular Biology, (1986) and Sambrook et al., Molecular Cloning: A Laboratory Manual, 2nd Ed., (see above).

Representative examples of appropriate hosts include bacterial cells such as E. coli, fungal cells such as Saccharomyces, insect cells such as Drosophila S2 and Spodoptera Sf9 cell, animal cells such as CHO, COS, HeLa, C127,3T3, BHK, 293 and Bowes melanoma cells; and plant cells.

The appropriate DNA sequence may be inserted into the expression system by any of a variety of well-known and routine techniques, such as, for example, those set forth in Sambrook et al., Molecular Cloning: A Laboratory Manual, 2nd Ed, (supra).

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Polypeptides of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Most preferably, high performance liquid chromatography is employed for purification. Well known techniques for refolding protein may be employed to regenerate active conformation when the polypeptide is denatured during isolation and or purification.

This invention is also comprises the use of the ykuR polynucleotides of the invention as diagnostic reagents. Detection of ykuR in a eukaryote, particularly a mammal, and especially a human, will provide a diagnostic method for diagnosis of a disease. Eukaryotes, particularly mammals, and especially humans, particularly those infected or suspected to be infected with an organism comprising the ykuR gene may be detected at the nucleic acid level by a variety of techniques. In this connection, nucleic acids for diagnosis may be obtained from an infected individual's cells and tissues, such as blood, muscle, cartilage, and skin. Genomic DNA may be used directly for detection or may be amplified enzymatically by using PCR or other amplification technique prior to analysis. A process for diagnosing disease bacterial infections may comprise determining from a sample derived from an individual a diagnostically significant level of polynucleotide of the invention. The level of ykuR polynucleotide can be measured using any on of the methods well known in the art for the quantation of polynucleotides, such as, for example, amplification, PCR, RT-PCR, RNase protection, Northern blotting and other hybridization methods.

The polypeptides of the invention, or cells expressing them can be used as an immunogen to produce antibodies immunospecific for such polypeptides. “Antibodies” as used herein includes monoclonal and polyclonal antibodies, chimeric, single chain, simianized antibodies and humanized antibodies, as well as Fab fragments, including the products of an Fab immunolglobulin expression library.

Antibodies generated against the polypeptides of the invention can be obtained by administering epitope bearing polypeptides of the invention or cells containing them to an animal, preferably a nonhuman, using routine protocols. For preparation of monoclonal antibodies, any technique known in the art that provides antibodies produced by continuous cell line cultures can be used. Examples include various techniques, such as those in Kohler, G. and Milstein, C., Nature 256: 495-497 (1975); Kozbor et al., Immunology Today 4: 72 (1983); Cole et al., pg. 77-96 in MONOCLONAL ANTIBODIES AND CANCER THERAPY, Alan R. Liss, Inc. (1985). Such antibodies against ykuR-polypeptide may be employed to treat infections, particularly bacterial infections.

The use of a polynucleotide of the invention in genetic immunization will preferably employ a suitable delivery method such as direct injection of plasmid DNA into muscles (Wolff et al., Hum Mol Genet 1992, 1: 363, Manthorpe et al., Hum. Gene Ther. 1963: 4, 419), delivery of DNA complexed with specific protein carriers (Wu et al., J Biol Chem. 1989: 264, 16985), coprecipitation of DNA with calcium phosphate (Benvenisty & Reshef, PNAS USA, 1986: 83, 9551), encapsulation of DNA in various forms of liposomes (Kaneda et al., Science 1989: 243, 375), particle bombardment (Tang et al., Nature 1992, 356: 152, Eisenbraun et al., DNA Cell Biol 1993, 12: 791) and in vivo infection using cloned retroviral vectors (Seeger et al., PNAS USA 1984: 81, 5849).

Also within the scope of the invention is a method of screening compounds to identify those which inhibit the action of ykuR polypeptides or polynucleotides, then selecting those which are bacteriostatic and/or bactericidal in one or more bacterial cell assays. The method of screening may involve high-throughput techniques. For example, to screen for inhibitors, a synthetic reaction mix, a cellular compartment, such as a membrane, cell envelope or cell wall, or a preparation of any thereof, comprising ykuR polynucleotide or polypeptide and a labeled substrate or ligand of such polynucleotide or polypeptide is incubated in the absence or the presence of a candidate molecule that may be a ykuR inhibitor. The ability of the candidate molecule to inhibit expression of the ykuR polypeptide or its enzymic activity is reflected in decreased binding of the labeled ligand or decreased production of product from such substrate. Molecules that bind gratuitously, i.e., without inducing the effects of ykuR polypeptide are most likely to be good inhibitors. Detection of the rate or level of production of product from substrate may be enhanced by using a reporter system. Reporter systems that may be useful in this regard include but are not limited to colorimetric labeled substrate converted into product, a reporter gene that is responsive to changes in ykuR polynucleotide or polypeptide activity, and binding assays known in the art.

Another aspect of the invention relates to a method for inducing an immunological response in an individual, particularly a mammal which comprises inoculating the individual with ykuR polypeptide, or a subsequence or variant thereof, adequate to produce antibody and/or T cell immune response to protect said individual from bacterial infection. Yet another aspect of the invention relates to a method of inducing immunological response in an individual which comprises delivering to such individual a nucleic acid vector to direct expression of ykuR, or a fragment or a variant thereof, for expressing ykuR, or a fragment or a variant thereof in vivo in order to induce an immunological response, such as, to produce antibody and/or T cell immune response, including, for example, cytokine-producing T cells or cytotoxic T cells, to protect said individual from disease, whether that disease is already established within the individual or not.

One way of administering the gene is by accelerating it into the desired cells as a coating on particles or otherwise. Such nucleic acid vector may comprise DNA, RNA, a modified nucleic acid, or a DNA/RNA hybrid.

Moreover, the co-protein may act as an adjuvant in the sense of providing a generalized stimulation of the immune system. The co-protein may be attached to either the amino or carboxy terminus of the first protein.

Also provided by this invention are compositions, particularly vaccine compositions, and methods comprising the polypeptides or polynucleotides, or subsequences or variants thereof, of the invention together with a suitable carrier.

### Examples

Cloning of B subtilis ykuR Polynucleotide.

The following oligonucleotide sequences were synthesized as forward and reverse primers:

Forward primer:

(SEQ ID No: 3)

5′-GACGACGACCATATGAAGATAGAGGAGCTCATCGC
Reverse primer:

(SEQ ID No: 4)

5′-GACCGGATCCTAGAGTGCTCATTTTATGG

The above primer pairs were used to PCR amplify ykuR polynucleotide sequence from the B. subtilis genomic DNA using pfu polymerase. In so doing a NdeI restriction site (underlined in forward primers) was created immediately prior to the ATG start of translation (bold in forward primers) and a BamHI restriction site (underlined in reverse primer) was created after the end of the polynucleotide coding sequence. Isolation of the amplified polynucleotide sequences was confirmed by agarose gel electrophoresis and the polynucleotide was digested with NdeI and BamHI restriction enzymes according to manufacturers instructions. In the case of ykuR the NdeI restriction digest was carried out for only 30 minutes to partially digest the polynucleotide which contains an internal NdeI restriction site (underlined in nucleotide sequence) the full length ykuR gene product could be separated from internally digested gene product by agarose gel electrophoresis.

Digested polynucleotide ykuR gene product was ligated into predigested NdeI and BamHI pET24a and the polynucleotide sequence SEQ ID No: 1 confirmed by dideoxy dye terminator sequencing (Applied Biosystems).

All enzyme reactions were performed according to manufacturers instructions (Promega).

Expression of ykuR Polypeptide

The ykuR polynucleotide was cloned into pET24a plasmid which was transformed into the E. coli expression strain BL21 and the plasmid selected for, using 50 μg/ml kanamycin. A single colony was used to inoculate a 10 ml overnight culture in 2xYT media. This culture was used to inoculate a prewarmed baffle flask containing 500 ml of 2xYT media. Cells were grown at 37° C. with shaking until an OD600 nm of 0.4 was reached. At this point IPTG was added to a final concentration of 0.4 mM and the flask incubated for a further 3 hours at 25° C. with shaking. Bacteria were harvested by centrifugation at 5000 g for 10 minutes.

The cell pellet was resuspended and washed once in ice-cold PBS and stored at −70° C.

Purification of ykuR Polypeptide

The cell pellet was resuspended in 27 ml of buffer and sonicated 5×40 second bursts on ice using medium size probe and 22 micron amplitude setting. The cell debris was pelleted by centrifugation at 15000 g for 25 minutes at 4° C.

The soluble fraction was loaded onto a pre-equilibrated (with buffer A^{*1}) Q-Sepharose anion exchange column (1×9 cm) at 3 ml/min. at RT. The column was washed with the same buffer until the absorbence at A280 returned to baseline and was eluted with a linear gradient of 0-1M NaCl in buffer A. The protein eluted at around 0.52M NaCl as determined by SDS-PAGE. The fractions containing the majority of the protein were pooled (30 ml) and concentrated down to approximately 1.5 ml using the Millipore ultrafiltration Centricon device with a 5 kDa cut-off.

The 1.5 ml protein sample was loaded onto a Superdex 200 (1.5×70 cm) and eluted at 1 ml/min in 20 mM HEPES, 20 mM NaCl, pH6.8 buffer at 4° C. The fractions were analysed by SDS-PAGE and the cleanest fractions were pooled, concentrated down to 2 ml and diafiltered into buffer A.

A final polishing step was carried out on a preparative 8 ml MonoQ column at RT. The 2 ml sample was loaded onto the column and a shallow salt gradient (between 0.4 and 0.6M NaCl) was used to separate the minor contaminants away from the YkuR protein. The fractions were analysed by SDS-PAGE and the cleanest fractions were pooled and assayed.

The protein concentration was determined by A280, using a calculated extinction coefficient of 33900 M⁻¹ cm⁻¹.

Using this protocol the purity of YkuR polypeptide was estimated to be greater than 95%.

*1 20 mM HEPES. PH 6.8

### Sequencing of YkuR Polypeptide

Purified YkuR protein was separated by SDS-PAGE and electroblotted onto ProBlot PVDF membrane (100 Volts for one hour). The blot was washed three times in ddH20 for 30 minutes, dried and stained with Sulphorhodamine B. The 41 kDa YkuR band was excised and analysed by pulsed liquid N-terminal sequencing on an Applied Biosystems procise 494 automated sequencer.

Testing ykuR B. subtilis for Essentiality.

### A. Regulatable Gene Expression

The pmutin4 plasmid (Vagner et. al. 1998) was used to replace the wild-type promoter of ykuR with the IPTG regulatable promoter pSpaC—strain KO44G. In a control experiment the pSpaC promoter was integrated into the B. subtilis genome immediately after the full-length ykuR gene—strain KO44E.

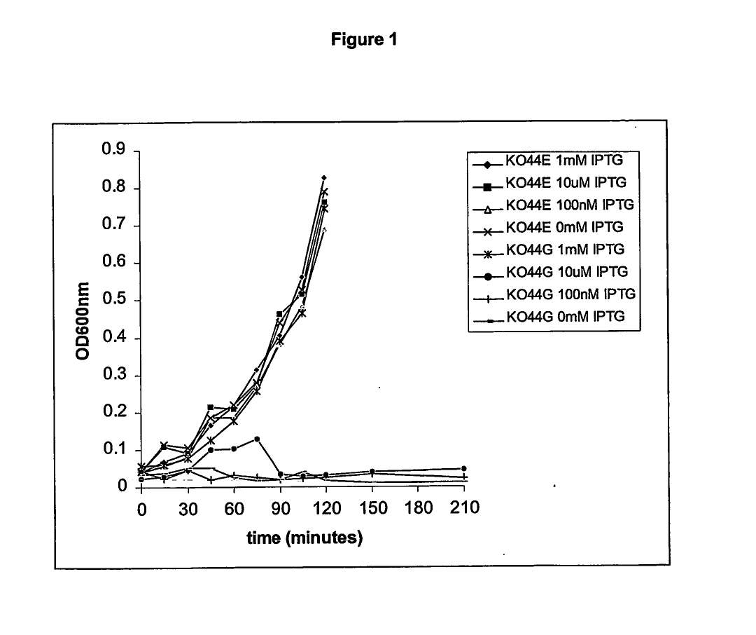

The pmutin4 plasmid carries a copy of the LacI gene whose product is known to bind to the pSpaC promoter in the absence of IPTG and prevent transcription of downstream genes. However, in the absence of IPTG, both of these strains displayed wild-type growth. It was surmised that this was due to incomplete suppression of the pSpaC promoter by LacI. Therefore, plasmid p65 was introduced to the KO44G and KO44E strains (Petit et al, (1998) Mol Microbiol, 29: 261-273). This plasmid is based on the pUB110 plasmid of Gram-positive bacteria and carries the LacI gene under the control of the PenP promoter for constitutive expression. Introduction of these additional copies of the LacI gene conferred IPTG dependence on KO44G but not KO44E, directly illustrating the importance of ykuR to B. subtilis cells (FIG. 1).

### Method

Strains were growth overnight in LB broth containing 10 μg/ml kanamycin (selects for p65), 0.3 μg/ml erythromycin (selects for pmutin4 integration) and 1 mM IPTG. A fresh culture was innoculated by diluting the overnight culture 1:100 and the strains grown until they reached log phase growth (OD600 nm of 0.4). The bacteria were washed twice with prewarmed LB media containing no IPTG and used to inoculate 50 ml cultures by diluting 1:250 into LB media containing erythromycin and kanamycin with varying quantities of IPTG. Measuring the optical density at 600 nm monitored growth of the strains at 37° C.

B. Lethality of ykuR Suppression

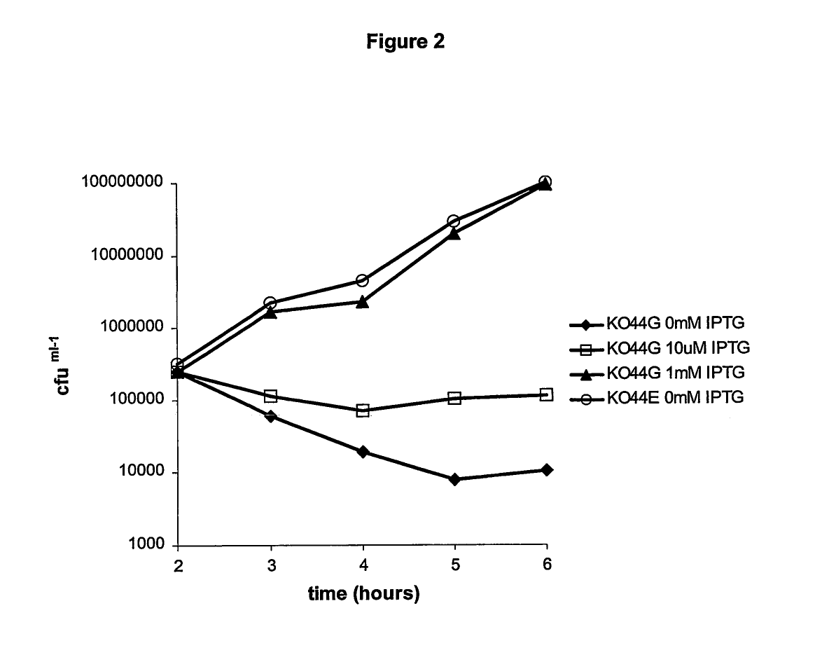

Strains KO44G and KO44E were grown overnight in LB broth containing 10 μg/ml kanamycin (selects for p65), 0.3 μg/ml erythromycin (selects for pmutin4 integration) and 1 mM IPTG. A fresh culture was innoculated by diluting the overnight culture 1:100 and the strains grown until they reached log phase growth (OD600 nm of 0.4). The bacteria were washed twice with prewarmed LB media containing no IPTG, innoculated into prewarmed LB media containing erythromycin and kanamycin but no IPTG at a starting OD600 nm of 0.02 and allowed to grow over a period of two hours to an OD600 nm of 0.2 to deplete ykuR levels. This culture was diluted 1:100 in prewarmed LB media containing erythromycin and kanamycin and IPTG added at various concentrations. The number of viable bacteria over time was calculated by plating dilutions of these cultures on LB plates containing 1 mM IPTG. The results are shown in FIG. 2. These results suggest that lack of ykuR produces a bactericidal phenotype in B. subtilis and hence anti-bacterial agents which target ykuR are likely to be bactericidal in nature.

### YkuR Surrogate Assay

A fluorometric assay for YkuR and its homologues has been developed using sodium hippurate (benzoyl glycine) as a substrate. Cleavage by YkuR releases free glycine which can be detected using F-phthaldialdehyde. The assay is performed in a total volume of 100 Fl per well in black 96 well microtitre plates. 40 Fl of YkuR protein 20 Fg/ml in 10 mM Hepes pH 6.8 containing 0.5 mM dithiothreitol and 10 mM potassium chloride) is added to the appropriate wells of a 96-well plate. Appropriate concentrations of test compounds dissolved in 20 Fl of 100% DMSO are added, followed by 40 Fl of hippuric acid (250 mM in 50 mM Hepes pH 6.8 containing 0.05% Brij 35). Control wells lack either enzyme or test compound. The reactions are incubated for 1 hour at 37 EC. Following incubation 100 Fl of OPA Reagent (20 mM F-phthaldialdehyde in 50 mM borate buffer pH 9.5 containing 0.1% $-mercaptoethanol) is added to each well. The fluorescence at 460 nm is measure with an SLT Fluostar fluorometer using 355 nm excitation.

### Alternative Assays

### Hypersensitisation

The Escherichia coli D22 strain was used to screen for antibiotics, which inhibit LpxC through hypersensitisation. Mutation in the LpxC gene of the D22 strain allows compounds greater access to enter the bacteria and reduces the activity of LpxC within the bacteria which needs to be overcome in order to kill the organism.

In a similar fashion, reduced expression through changing the promoter of YkuR (described above) or generation of mutations within YkuR may hypersensitise a bacterium to inhibitors of YkuR. Screening of such strains against a panel of inhibitors could be used as an assay.

### Reporter Assays

Reduced expression through changing the promoter of YkuR (described above) or activity of YkuR may evoke a transcriptional response by the bacterium. The expression of several genes may be altered. Insertion of a reporter gene such as B-galactosidase into the genome or into a plasmid vector such that it becomes regulated by genes responding to a change in the levels/activity of YkuR, could be used as an alternative whole cell assay to identify inhibitors of YkuR.

Metalloprotease Inhibitor Inhibits ykuR Polypeptide Activity

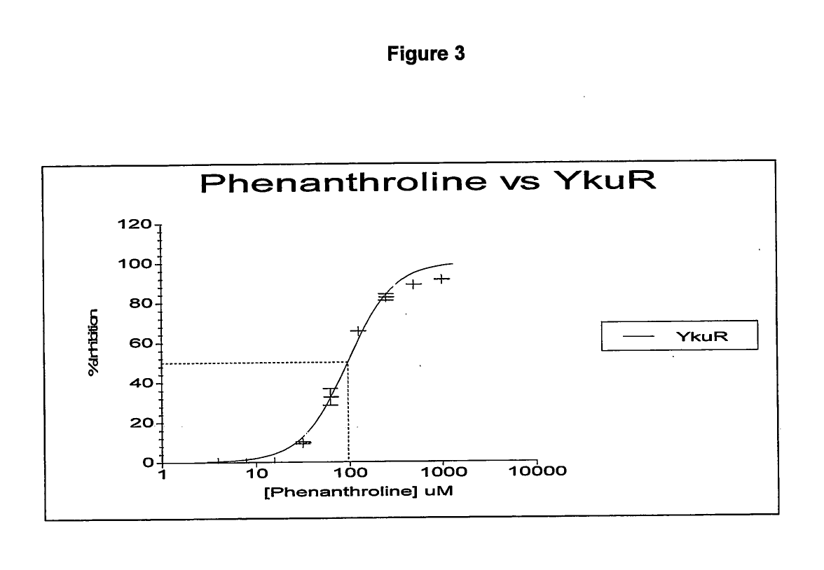

Assay using purified YkuR protein expressed in E. coli as described above. Phenanthroline, a known inhibitor of metalloprotease enzyme activity, was added at increasing concentrations to inhibit metalloenzyme activity. IC50 was 96 μM, see FIG. 3.

Conservation OF ykuR ORF.

BLAST searches of pathogenic bacterial, yeast and human genome and EST databases were used to identify gene homologues of ykuR (Table 3). The bacterial homologues identified have a greater than 50% sequence homology to YkuR, and are to be regarded as variants thereof.

TABLE 3

| | Gene | Accession | BLAST | % iden- | % simi- |
|---|---|---|---|---|---|
| Organism | Name | number | score | tity | larity |
| B. subtilis | ykuR | 034916 | 0.0 | 100 | |
| | ytnL | O34980 | 4E−66 | 39 | 54 |
| | yxeP | P54955 | 2E−60 | 36 | 55 |
| | yhaA | O07598 | 7E−53 | 35 | 50 |
| S. aureus Mu50 | | BAB58491.1 | 2E−53 | 34 | 53 |
| | | BAB56264.1 | 1E−52 | 33 | 50 |
| | | BAB56711.1 | 1E−50 | 32 | 51 |
| E. faecalis | | Gnl | 8E−96 | 46 | 62 |
| | | TIGR_1351 | | | |
| S. pnuemoniae | | AAK76155.1 | 8E−96 | 46 | 63 |
| S. cerevisae | | none | | | |
| Human | | none | | | |

ykuR is well conserved throughout bacteria including Staphylococcus aureus, Streptococcus and Enterococcus sp. No homologue (BLAST score <1E−10) could be identified in the S. cerevisiae, human or rodent genome and EST databases. It has been demonstrated that in the YkuR surrogate assay activity can be observed with purified S. pnuemoniae YkuR and B. subtilis YxeP.

1. An isolated polynucleotide of SEQ ID:1, or a subsequence or variant thereof.

2. An isolated polynucleotide that is complementary to the polynucleotide of claim 1.

3. An expression vector comprising the polynucleotide of claim 1 or claim 2.

4. A host cell comprising the vector of claim 3.

5. An isolated polypeptide of SEQ ID:2, or a subsequence or variant thereof.

6. A process for producing a polypeptide comprising expressing from the host cell of claim 4 a polypeptide encoded by said polynucleotide.

7. A process for producing a ykuR polypeptide or fragment comprising culturing a host of claim 4 under conditions sufficient for the production of said polypeptide.

8. A process for diagnosing a disease related to activity of the polynucleotide of claim 1 or activity or expression of a polypeptide of claim 5 in an individual comprising analyzing for the presence or amount of said polynucleotide or polypeptide in a sample derived from the individual.

9. A method for identifying antibacterially active compounds which interact with and inhibit an activity of a polypeptide of claim 5 comprising
contacting a composition comprising the polypeptide with compounds to be screened under conditions to permit interaction between the compounds and the polypeptide to assess the interaction of a compounds,
such interaction being associated with a second component capable of providing a detectable signal in response to the interaction of the polypeptide with the compound,
selecting compounds which interact with and inhibit an activity of the polypeptide by detecting the presence or absence of a signal generated from the interaction of the compounds with the polypeptide,
then testing the selected compounds for antibacterial activity in a bacterial cell assay, and selecting compounds active in such assay.

10. A method for inducing an immunological response in a mammal which comprises inoculating the mammal with ykuR polynucleotide of claim 1 or polypeptide of claim 5 adequate to produce antibody and/or T cell immune response to protect said animal from disease.

11. A method of inducing immunological response in a mammal which comprises delivering a nucleic acid vector to direct expression of ykuR polypeptide of claim 5 in vivo in order to induce an immunological response to produce antibody and/or T cell immune response to protect said animal from disease.
